(19) United States
(12) Patent Application Publication
(10) Pub. No.: US 2022/0280660 A1
(43) Pub. Date: Sep. 8, 2022

(54) COMPOSITION FOR ULTRASOUND CONTRAST AGENT, ULTRASOUND CONTRAST AGENT AND PREPARATION METHOD THEREOF

(71) Applicant: Nanjing Transcend Vivoscope Bio-Technology Co., LTD, Nanjing (CN)

(72) Inventors: Wenyu GUO, Nanjing (CN); Shuo HUANG, Nanjing (CN); Feihong DONG, Nanjing (CN); Jue ZHANG, Nanjing (CN)

(73) Assignee: Nanjing Transcend Vivoscope Bio-Technology Co., LTD, Nanjing (CN)

(21) Appl. No.: 17/752,104

(22) Filed: May 24, 2022

Related U.S. Application Data
(63) Continuation of application No. PCT/CN2021/084077, filed on Mar. 30, 2021.

(30) Foreign Application Priority Data
Jun. 28, 2020 (CN) 202010597801.6

(51) Int. Cl. A61K 49/22

(52) U.S. Cl. CPC A61K 49/221; A61K 49/223

(57) ABSTRACT

Disclosed are a composition for ultrasound contrast agent, an ultrasound contrast agent, and a preparation method thereof. The composition for ultrasound contrast agent includes a lipid, a stabilizer and an acoustic-induced deformation material; relative to 100 parts by weight of the lipid, the content of the stabilizer is 20 to 100 parts by weight, and the content of the acoustic-induced deformation material is 1 to 15 parts by weight; and the acoustic-induced deformation material is deformed under a specific acoustic wave, and the characteristic response frequency of the acoustic-induced deformation material is 0.01 MHz to 50 MHz. The microbubble ultrasound contrast agent has better stability, thereby it circulates in vivo for a longer time, and has lower mechanical index, so that the inertial cavitation occurs under a low-energy ultrasonic wave.

### CROSS-REFERENCE TO RELATED APPLICATIONS

This application is a continuation of International Application No. PCT/CN2021/084077 filed on Mar. 30, 2021, which claims priority to Chinese Patent Application No. 202010597801.6 filed on Jun. 28, 2020. The disclosures of the above-mentioned applications are hereby incorporated by reference in their entireties.

### TECHNICAL FIELD

The disclosure relates to the field of biomedicine, in particular to a composition for ultrasound contrast agent, an ultrasound contrast agent including the composition for ultrasound contrast agent, a preparation method of an ultrasound contrast agent and an ultrasound contrast agent prepared by the preparation method.

### BACKGROUND

Although the survival rate of cancer has improved overall, chemotherapy drugs are prone to adverse effects and drug resistance. In addition, solid tumors have sparse blood vessels and no lymphatic drainage, which lead to high tissue fluid pressure and poor drug delivery in the tumors. As a result, cancer remains the world's second leading cause of death. Therefore, it is urgent to develop effective and safe strategies for targeted delivery of chemotherapeutic drugs. Ultrasonic diagnosis is a low-cost, non-invasive and non-radiation real-time imaging technology, which is the most widely-available imaging method used in clinical and scientific research at present. Ultrasound Contrast Agents (UCAs) is a kind of diagnostic reagent that can significantly enhance the detection signal and detection sensitivity of medical ultrasound after intravenous injection, and is a kind of microbubble solution with a diameter of about 1 μm to 10 μm. The inertial cavitation occurs upon rupture of a microbubble under a high-energy ultrasonic wave, so that the microbubble loads drug to blast at the target area and release the drug locally, thereby exhibiting the therapeutic effect of targeted drug delivery. In addition, the cavitation of the microbubble locally generates micro-flow and shear force, thereby stimulating the channels between the pores of the endothelial cell membrane and cells to be opened, and facilitating the delivery of the therapeutic drug into the cell. This makes Ultrasonic Targeted Microbubble Destruction (UTMD) attract more and more attention as a promising non-invasive targeted drug delivery technology.

At present, the guidance and monitoring for the UTMD technology are mainly based on MRI with a spatial resolution of 1 mm and a temporal resolution of 1 s, which is not only expensive and complicated to operate, but also difficult to guide the position of microbubble blasting accurately and give real-time feedback of treatment effect, and even may lead to unnecessary tissue damages. A real-time ultrasound-guided UTMD system is urgently needed, which realizes the real-time imaging function and microbubble breaking function simultaneously based on the same ultrasonic imaging probe.

However, in order to improve the drug concentration of target and utilization rate of the drug, it is often needed to keep the drug-loaded ultrasound contrast agent circulating in vivo for a longer time (>6 min, even >10 min), i.e., the stability of the contract agent needs to be improved. But the improvement of the stability for the ultrasound contrast agent means that the ultrasonic energy for the microbubble blasting needs to be increased. It is difficult of real-time ultrasound-guiding UTMD based on the same ultrasonic imaging probe, because the ultrasonic imaging probe (MI<1.9) is hard to break the microbubbles.

Therefore, a low mechanical index drug-loaded ultrasound contrast agent that maintains a longer circulation time in vivo and produces inertial cavitation under a low-energy ultrasonic wave is urgently needed, so as to act as both contrast agent and drug delivery agent under the same ultrasonic imaging probe.

### SUMMARY

The object of the present disclosure is to solve the contradiction that the existing ultrasound contrast agent maintains a longer circulation time while the energy threshold needed by inertial cavitation is increased, and to provide a composition for ultrasound contrast agent, an ultrasound contrast agent including the composition for ultrasound contrast agent, a preparation method of an ultrasound contrast agent and an ultrasound contrast agent prepared by the preparation method. The microbubble ultrasound contrast agent obtained by the composition for ultrasound contrast agent according to the present disclosure has better stability, thereby the microbubble ultrasound contrast agent circulates in vivo for a longer time (>6 min, in the preferred embodiment it can be >10 min, even up to 15 min) and has a lower mechanical index (ultrasound mechanical index MI<1.5), so that the inertial cavitation occurs under a low-energy ultrasonic wave. Therefore, the microbubble ultrasound contrast agent obtained by the composition for ultrasound contrast agent according to the present disclosure act as both a contrast agent and a drug delivery agent simultaneously under the same ultrasonic imaging probe.

The inventors of the present disclosure have found that by introducing an acoustic-induced deformation material deformed under a specific sound field intensity, the diffusible stress concentration may be occurred on the surface of the ultrasound contrast agent under the ultrasound. The change of the local stress distribution on the surface of the ultrasound contrast agent will cause poor mechanical stability of the contrast agent significantly under the sound field, and the inertial cavitation is more likely to occur. Therefore, it is impossible to obtain a low mechanical index drug-loaded ultrasound contrast agent. The drug-loaded ultrasound contrast agent generates inertial cavitation and release a drug under a low mechanical index ultrasound (ultrasonic mechanical index MI<1.5) while ensuring a longer circulation time in vivo.

A first aspect of the present disclosure provides a composition for ultrasound contrast agent, which includes a lipid, a stabilizer and an acoustic-induced deformation material; wherein relative to 100 parts by weight of the lipid, the content of the stabilizer is 20 to 100 (e.g. 20, 30, 40, 50, 60, 70, 80, 90 or 100) parts by weight, and the content of the acoustic-induced deformation material is 1 to 15 (e.g. 1, 2, 3, 4, 5, 6, 7, 8, 9, 10, 11, 12, 13, 14 or 15) parts by weight; and wherein the acoustic-induced deformation material is deformed under the acoustic wave of a characteristic response frequency for the acoustic-induced deformation material, and the characteristic response frequency is 0.01 MHz to 50 MHz.

The specific ratio of the above lipid, stabilizer and acoustic-induced deformation material according to the present disclosure may achieve better effects. In order to further improve stability and reduce mechanical index, preferably, relative to 100 parts by weight of the lipid, the content of the stabilizer is 30 to 60 parts by weight, and the content of the acoustic-induced deformation material is 3 to 12 parts by weight; more preferably, relative to 100 parts by weight of the lipid, the content of the stabilizer is 42 to 50 (e.g. 42, 43, 44, 45, 46, 47, 48, 49 or 50) parts by weight, and the content of the acoustic-induced deformation material is 4 to 10 parts by weight.

Understandably, although the microbubble ultrasound contrast agent of the present disclosure may be used as a drug delivery agent, the composition for ultrasound contrast agent of the present disclosure may not include the drug based on the needs of production and transportation according to a specific embodiment.

According to another specific embodiment of the present disclosure, the composition for ultrasound contrast agent further includes a drug, relative to 100 parts by weight of the lipid, the content of the drug is 2 to 20 (e.g., 2, 3, 4, 5, 6, 7, 8, 9, 10, 11, 12, 13, 14, 15, 16, 17, 18, 19 or 20) parts by weight, more preferably 6 to 10 parts by weight.

In order to use the product of the present disclosure as an ultrasound contrast agent specifically, preferably the acoustic-induced deformation material is sensitive in the range of medical diagnostic ultrasonic frequency (generally 1 MHz to 30 MHz). Preferably, the characteristic response frequency of the acoustic-induced deformation material is 1 MHz to 30 MHz (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30), more preferably 2 MHz to 20 MHz. The preferred range of the characteristic response frequency for the acoustic-induced deformation material is not strictly required. According to the specific acoustic-induced deformation material in clinical application, a corresponding characteristic response frequency is selected as the center frequency of the ultrasound.

In the present disclosure, the term “acoustic-induced deformation material” is rarely used or never used in the art because of this kind of material has received little attention and research at present and has never been used in the field of biomedicine, especially in the ultrasound contrast agent. The term “acoustic-induced deformation material” defined by the inventors of the present disclosure has a meaning of “the material does deformation under the specific acoustic wave (frequency, intensity and the like)”, similar with the term “photostrictive material”, wherein the deformation are various morphological changes, such as, expansion, contraction, bending, and the like. The characteristic response frequency of a specific material is a specific frequency of acoustic wave which make the specific material deformed. A material can be used in the ultrasound contrast agent of the present disclosure when its characteristic response frequency falls between the range of the medical diagnostic ultrasonic frequency. In use, firstly adjusting the ultrasonic frequency to non-characteristic response frequency below to perform routine contrast-enhanced ultrasound; then, adjusting the ultrasonic frequency to the characteristic response frequency (with or without enhancing the mechanical index). Under the influence of the acoustic-induced deformation material, the cavitation occurs upon rupture of the microbubble, and the force caused by the cavitation would release a therapeutic drug and promote its delivery into cells, so that the inertial cavitation occurs and the drug is released under a low mechanical index ultrasonic wave. Therefore, the ultrasound contrast agent of the present disclosure act as both a contrast agent and a drug delivery agent simultaneously under the same ultrasound imaging probe.

In the present disclosure, a material satisfying the above conditions may be used as the acoustic-induced deformation material of the present disclosure, preferably, the acoustic-induced deformation material is any one or more selected from the group consisting of: poly N-isopropylacrylamide (PNIPAm), poly vinyl caprolactam, hematoporphyrin, photofrin (Photofrin II), mesoporphyrin, sodium porphyrin (DVDMS), gallium porphyrin (ATX-70), hydrophilic chlorin derivative (ATX-S10), protoporphyrin, copper protoporphyrin, tetraphenylporphyrin tetrasulfonate, phoeophorbide a, photosensory proteins, adriamycin, chlorin e6, bengal red, erythrosin B, curcumin, methylene blue, tenoxicam, piroxicam, artemether (LEA) and water-soluble chlorin derivative (PAD-S31); more preferably, the acoustic-induced deformation material is any one or more selected from the group consisting of: poly N-isopropylacrylamide, poly vinyl caprolactam and artemether.

In the present disclosure, the lipid may be a lipid commonly used in the art for the ultrasound contrast agent. Preferably, the lipid is phospholipids. In order to have a better synergistic effect with other components in the composition for the ultrasound contrast agent of the present disclosure, more preferably, the lipid is any one or more selected from the group consisting of: 1,2-distearoyl-sn-glycero-3-phosphocholine (DSPC), distearoylphosphatidylethanolamine (DSPE), dipalmitoylphosphatidylcholine (DPPC), 1,2-bis(diphenylphosphine)ethane (DPPE), and distearoylphosphatidylethanolamine-polyethylene glycol 2000 (DSPE-PEG2000).

In the present disclosure, preferably the lipid is a combination of two or more substances. According to a preferred embodiment, the lipid consists of a first lipid and a second lipid in a weight ratio of 1:0.05 to 1:0.5:(more preferably 1:0.1 to 1:0.3), wherein the first lipid is DPPC and/or DSPC, and the second lipid is DSPE and/or DPPE.

In the present disclosure, the stabilizer may be a stabilizer commonly used in the art for the ultrasound contrast agent. For example, the stabilizer is any one or more selected from the group consisting of: polyoxypropylene polyoxyethylene block polyether (Pluronic), polyethylene glycol 4000 (PEG4000), polyethylene glycol 2000 (PEG2000), polyethylene glycol 1400 (PEG1400), polyethylene glycol 40s (PEG40s) and polysorbate-80.

In the present disclosure, preferably, the stabilizer is Pluronic, or the stabilizer is a combination of Pluronic with at least one of the PEG4000, PEG2000, PEG1400 and PEG40s in a weight ratio of 1:0.5 to 1:0.8.

In the composition of the present disclosure, these components may exist alone, or may have been pre-combined. For example, a commercially available raw material DSPE-PEG2000 is a pre-combined form of the lipid DSPE and the stabilizer PEG2000. In the specific embodiment of the present disclosure, the calculation method of dosage is calculating the dosage of DSPE and PEG2000 separately when using the raw material.

In the present disclosure, the drug may be various drugs as required for actual treatment. Preferably, the drug is any one or more selected from the group consisting of: paclitaxel, hydroxycamptothecin, adriamycin, bleomycin, gecitabine, vinorelbine, lentinan, docetaxe and elemene.

The second aspect of the present disclosure provides an ultrasound contrast agent, wherein the ultrasound contrast agent includes the composition for ultrasound contrast agent according to the first aspect of the present disclosure, or the ultrasound contrast agent is prepared by the composition for ultrasound contrast agent.

According to a specific embodiment of the present disclosure, the ultrasound contrast agent includes a large number of gas-filled microbubbles.

In this specific embodiment, preferably, the particle size of the gas-filled microbubbles is 0.1 μm to 10 μm (e.g., 0.1, 0.2, 0.3, 0.4, 0.5, 0.6, 0.7, 0.8, 0.9, 1, 2, 3, 4, 5, 6, 7, 8, 9 or 10), more preferably 0.5 μm to 2 μm.

Preferably, the gas in the gas-filled microbubbles is an inert gas and may be commonly used in the art for microbubble ultrasound contrast agent, such as, any one or more selected from the group consisting of: perfluoropropane, perfluorobutane and sulfur hexafluoride. The shell of the gas-filled microbubbles includes the composition for ultrasound contrast agent according to the first aspect of the present disclosure.

According to another specific embodiment of the present disclosure, the ultrasound contrast agent may not contain gas-filled microbubbles for convenience of storage and transportation. Generally, in the art, the ultrasound contrast agent in this state is called film-forming solution for ultrasound contrast agent. The ultrasound contrast agent that includes a large number of gas-filled microbubbles and may be used in clinical application is obtained by the film-forming solution for ultrasound contrast agent through putting a degree of mechanical force on the film-forming solution for ultrasound contrast agent. The specific operation mode may be as the step (4) in the method according to the third aspect of the present disclosure, or be the other common method in the art.

In the present disclosure, the term “ultrasound contrast agent” also includes a film-forming solution for ultrasound contrast agent.

According to a specific embodiment of the present disclosure, the ultrasound contrast agent consists of a continuous phase and a dispersed phase. The continuous phase may be a conventional continuous phase used to prepare an ultrasound contrast agent in the field, such as phosphate (PBS) buffer solution. The dispersed phase includes the composition for ultrasound contrast agent according to the first aspect of the present disclosure, and may be the gas-filled microbubbles (i.e., forming an ultrasound contrast agent) or form a film-forming solution for ultrasound contrast agent.

A third aspect of the present disclosure provides a method for preparing the ultrasound contrast agent according to the second aspect of the present disclosure, wherein the method includes the following steps:
(1) mixing the composition for ultrasound contrast agent according to the first aspect of the present disclosure with a solvent to obtain solution A;
(2) rotary evaporating the solution A in a first water bath until a thin film is formed; and,
(3) adding a hydration solution into the material obtained by step (2) and rotary evaporating the resulting hydration solution in a second water bath until the thin film is dissolved to obtain solution B; and optionally,
(4) pumping a gas into the solution B, conducting ultrasonic cavitation to form gas-filled microbubbles.

In step (1), the choice of the solvent is not particularly limited, as long as the composition for ultrasound contrast agent according to the first aspect of the present disclosure can be dissolved in it without any chemical reaction. For example, the solvent is chloroform.

In step (2), the solution A is treated by a thin film emulsification method, specifically, rotary evaporating the solution A in a first water bath until a film is formed. Preferably, the rotary evaporation temperature of the first water bath is 45° C. to 70° C., more preferably 50° C. to 60° C.; preferably, the rotary evaporation pressure of the first water bath is negative pressure, such as 0.05 Mpa to 0.5 Mpa; the rotary evaporation time of the first water bath is not particularly limited, as long as the solvent may be removed basically, generally, it takes 10 min to 40 min relative to per 200 mg of the solution A.

In step (3), the volume ratio of the hydration solution and the solvent in step (1) is 1:(1-3).

In step (3), preferably, the hydration solution is a mixture of glycerol (i.e., glycerinum) and water or a mixture of glycerol (i.e., glycerinum) and acid-base buffer solution (such as PBS phosphate buffer solution); preferably, the volume content of the glycerol in the hydration solution is 10% to 30%.

In step (3), preferably, the rotary evaporation temperature of the second water bath is 45° C. to 70° C., more preferably 50° C. to 60° C.; the rotary evaporation time of the second water bath is not particularly limited, as long as the thin film may be completely dissolved, generally, it takes 10 min to 20 min relative to per 200 mg of the material.

The step (4) of the present disclosure may be optionally implemented or not according to actual need.

In the present disclosure, the solution B obtained by step (3) may be produced, sold and transported. The solution B is a specific embodiment of the microbubble ultrasound contrast agent of the present disclosure.

The method of the microbubble ultrasound contrast agent according to the present disclosure further includes step (4) when clinical application is needed.

In step (4), preferably, the conditions of the ultrasonic cavitation include: a power of 6 kW to 12 kW, and a time of 2 min to 15 min; more preferably, the conditions of the ultrasonic cavitation include: a power of 8 kW to 12 kW, and a time of 4 min to 10 min.

In step (4), the gas is that in the gas-filled microbubbles according to the second aspect of the present disclosure.

The obtained ultrasound contrast agent is the film-forming solution for ultrasound contrast agent without microbubbles when the method of the third aspect of the present disclosure does not includes step (4). The ultrasound contrast agent in this state is convenient to transport and store. However, in clinical application, the operation in step (4) should be carried out first to obtain the microbubble ultrasound contrast agent.

The obtained microbubble ultrasound contrast agent includes a large number of microbubbles, which may be used in clinic directly when the method of the third aspect of the present disclosure includes step (4).

Through the above technical solutions, compared with the prior art the present disclosure has at least the following advantages: the microbubble ultrasound contrast agent obtained by the composition for ultrasound contrast agent according to the present disclosure possess both higher stability and lower mechanical index, and may ensure a longer circulation time (>6 min, in the preferred embodiment it can be >10 min, even up to 15 min) in vivo while generates inertial cavitation and releases a drug under a low mechanical index ultrasound (ultrasound mechanical index MI<1.5). Therefore, the microbubble ultrasound contrast agent may act as both a contrast agent and a drug delivery agent simultaneously under the same ultrasonic imaging probe.

The endpoints of a range and any values disclosed herein are not limited to the precise ranges or values, which are to be understood to encompass values proximate to those ranges or values. For value ranges, the endpoints of each range, an endpoint of each range and an individual point value, and the individual point values may be combined with each other to yield one or more new value ranges which should be considered as particularly disclosed herein.

### BRIEF DESCRIPTION OF DRAWINGS

FIG. 1 is an image of the contrast agent II1 obtained in Example 1 under an optical microscope.

FIG. 2 is real-time ultrasonic images of the contrast agent II1 obtained in Example 1 before ultrasound-induced blast in rabbit kidney (a) and after ultrasound-induced blast in rabbit kidney (b).

### DETAILED DESCRIPTION OF EMBODIMENTS

The present disclosure will be described in detail below by the Examples. The described Examples of the present disclosure are only a part of the examples of the present disclosure, but not all of the examples. Based on the examples of the present disclosure, all other examples obtained by those of ordinary skill in the art without creative efforts shall fall within the protection scope of the present disclosure.

Unless otherwise specified, the materials used in the following Examples are all commercially available analytical grades.

### Preparation Example

The preparation example is used to prepare the hydration solution used in step (3), i.e., PBS-glycerol hydration solution. The preparation example is only a specific embodiment and not a limitation of the present disclosure. The specific process includes:

Weighing 0.097 g of KCl, 4.005 g of NaCl, 1.145 g of Na₂HPO₄. H₂O and 0.096 g of KH₂PO₄ to a beaker of 1 L, adding deionized water and making up to 500 ml to prepare phosphate buffered saline (PBS solution) for use. Weighting 85 ml of the PBS solution, adding 15 ml of glycerol, mixing well to obtain a PBS-glycerol hydration solution.

### Example 1

(I) Preparing a composition for ultrasound contrast agent, denoted as I1, including:

Lipid: 100 mg of dipalmitoylphosphatidylcholine (DPPC), 30 mg of distearoylphosphatidylethanolamine-polyethylene glycol 2000 (DSPE-PEG2000) (including 8 mg of lipid DSPE and 22 mg of stabilizer PEG2000);

Stabilizer: 30 mg of polyoxypropylene polyoxyethylene block polyether (Pluronic);

Acoustic-induced deformation material: 5 mg of poly N-isopropylacrylamide (PNIPAm);

Drug: 10 mg of paclitaxel.

(II) Preparing an ultrasound contrast agent, denoted as II1.

(1) Putting the composition for ultrasound contrast agent I1 into a round flask of 250 ml, and then adding 20 ml of chloroform, and fully mixing to make the solution clear and transparent;

(2) Removing the chloroform by a condition of the rotary evaporation temperature is 55° C. in water bath and the negative pressure is 0.05 MPa for 25 min to form a uniform thin film on the bottle of the flask;

(3) Adding 40 ml of the hydration solution obtained in the preparation example into the flask after step (2), continue rotary evaporating of 55° C. in water bath for 15 min to make the thin film dissolved completely, and then taking 5 ml of the resulting solution into a container of 15 ml;

(4) Putting the container into an ultrasonic pulverizer of 10 kW for 4 minutes while pumping perfluoropropane into the container to obtain an ultrasound contrast agent II1.

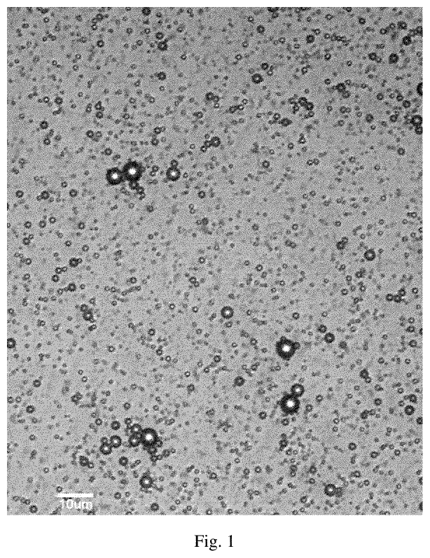

The obtained ultrasound contrast agent II1 is observed by an optical microscope and the result is shown in FIG. 1. It may be seen from the FIG. 1 that the contrast agent is covered in dense microbubbles with particle sizes of about 1 μm, and the particle size distribution of the microbubbles is narrow, and there are no obvious impurities in the solution, which can meet the imaging requirement for the ultrasound contrast agent.

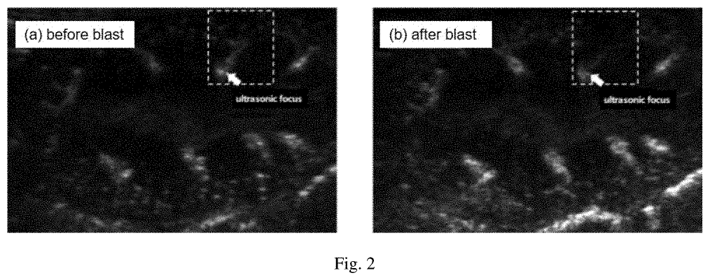

Taking a Japanese long-eared white rabbit as the experimental object, a peripheral vein channel is established through the marginal ear vein in the left ear of the rabbit, and a tri-branch tube is connected at the end of the catheter, and one of the channels is used for injecting the drug-loaded ultrasound contrast agent prepared by the present disclosure, and one channel for injecting normal saline solution. The Japanese white rabbit is anesthetized with 3% (40 mg/kg) sodium pentobarbital. After the rabbit is fully anesthetized, the right waist is depilated to facilitate renal angiography. The drug-loaded ultrasound contrast agent II1 (0.1 ml/kg) prepared by Example 1 is bolus injected through the rabbit marginal ear vein, and contrast-enhanced ultrasound and focused ultrasound, these two kinds of sequences are applied periodically on the same ultrasonic imaging probe. Recording the renal angiography of the rabbit under the contrast-enhanced ultrasound mode (center frequency: 8 MHz), and then breaking the microbubbles in artery specifically under the focused ultrasound mode (center frequency: 4 MHz (the frequency is the characteristic response frequency for PNIPAm), mechanical index: 0.8), switching the contrast-enhanced ultrasound mode immediately and recording the renal angiography of the rabbit after breaking the microbubbles specifically. The real-time ultrasonic images before and after the ultrasound-induced blast are shown in (a) and (b) of FIG. 2, respectively. The ultrasonic signal in the breaking point and the downstream of blood vessels of the breaking point are changed obviously in the images of before and after breaking the microbubbles specifically. The breaking point and the downstream of blood vessels of the breaking point are fully perfused before breaking the microbubbles, and the blood vessels boundary is clearly developed. Almost no echo signal may be seen in the breaking point and the downstream of blood vessels of the breaking point after breaking the microbubbles.

### Example 2

(I) Preparing a composition for ultrasound contrast agent, denoted as I2, including:

Lipid: 100 mg of DPPC, 10 mg of distearoylphosphatidylethanolamine (DSPE);

Stabilizer: 20 mg of polyethylene glycol 1400 (PEG1400), 30 mg of Pluronic;

Acoustic-induced deformation material: 10 mg of PNIPAm;

Drug: 10 mg of paclitaxel.

(II) Preparing an ultrasound contrast agent, denoted as II2.

(1) Putting the composition for ultrasound contrast agent I2 into a round flask of 250 ml, and then adding 20 ml of chloroform, and fully mixing to make the solution clear and transparent;

(2) Removing the chloroform by a condition of the rotary evaporation temperature is 55° C. in water bath and the negative pressure is 0.1 MPa for 30 min to form a uniform thin film on the bottle of the flask;

(3) Adding 40 ml of the hydration solution obtained in the preparation example into the flask after step (2), continue rotary evaporating of 55° C. in water bath for 15 min to make the thin film dissolved completely, and then taking 5 ml of the resulting solution into a container of 15 ml;

(4) Putting the container into an ultrasonic pulverizer of 10 kW for 6 minutes while pumping perfluoropropane into the container to obtain an ultrasound contrast agent II2.

### Example 3

(I) Preparing a composition for ultrasound contrast agent, denoted as I3, including:

Lipid: 100 mg of 1,2-distearoyl-sn-glycero-3-phosphocholine (DSPC), 10 mg of DSPE;

Stabilizer: 20 mg of polyethylene glycol 4000 (PEG4000), 30 mg of Pluronic;

Acoustic-induced deformation material: 5 mg of poly vinyl caprolactam;

Drug: 10 mg of paclitaxel.

(II) Preparing an ultrasound contrast agent, denoted as II3.

(1) Putting the composition for ultrasound contrast agent I3 into a round flask of 250 ml, and then adding 20 ml of chloroform, and fully mixing to make the solution clear and transparent;

(2) Removing the chloroform by a condition of the rotary evaporation temperature id 55° C. in water bath and the negative pressure is 0.01 MPa for 20 min to form a uniform thin film on the bottle of the flask;

(3) Adding 40 ml of the hydration solution obtained in the preparation example into the flask after step (2), continue rotary evaporating of 55° C. in water bath for 15 min to make the thin film dissolved completely, and then taking 5 ml of the resulting solution into a container of 15 ml;

(4) Putting the container into an ultrasonic pulverizer of 10 kW for 8 minutes while pumping perfluoropropane into the container to obtain an ultrasound contrast agent II3.

### Example 4

(I) Preparing a composition for ultrasound contrast agent, denoted as I4, including:

Lipid: 100 mg of DPPC, 10 mg of 1,2-bis(diphenylphosphine)ethane (DPPE);

Stabilizer: 20 mg of polyethylene glycol 40s (PEG40s), 30 mg of Pluronic;

Acoustic-induced deformation material: 10 mg of poly vinyl caprolactam;

Drug: 10 mg of paclitaxel.

(II) Preparing an ultrasound contrast agent, denoted as II4.

(1) Putting the composition for ultrasound contrast agent I4 into a round flask of 250 ml, and then adding 20 ml of chloroform, and fully mixing to make the solution clear and transparent;

(2) Removing the chloroform by a condition of the rotary evaporation temperature is 55° C. in water bath and the negative pressure is 0.15 MPa for 30 min to form a uniform thin film on the bottle of the flask;

(3) Adding 40 ml of the hydration solution obtained in the preparation example into the flask after step (2), continue rotary evaporating of 55° C. in water bath for 15 min to make the thin film dissolved completely, and then taking 5 ml of the resulting solution into a container of 15 ml;

(4) Putting the container into an ultrasonic pulverizer of 10 kW for 4 minutes while pumping perfluoropropane into the container to obtain an ultrasound contrast agent II4.

### Example 5

(I) Preparing a composition for ultrasound contrast agent, denoted as I5, including:

Lipid: 100 mg of DSPC, 30 mg of DSPE-PEG2000 (including 8 mg of lipid DSPE and 22 mg of stabilizer PEG2000);

Stabilizer: 30 mg of Pluronic;

Acoustic-induced deformation material: 5 mg of artemether (LEA);

Drug: 10 mg of paclitaxel.

(II) Preparing an ultrasound contrast agent, denoted as II5.

(1) Putting the composition for ultrasound contrast agent I5 into a round flask of 250 ml, and then adding 20 ml of chloroform, and fully mixing to make the solution clear and transparent;

(2) Removing the chloroform by a condition of the rotary evaporation temperature is 55° C. in water bath and the negative pressure is 0.2 MPa for 30 min to form a uniform thin film on the bottle of the flask;

(3) Adding 40 ml of the hydration solution obtained in the preparation example into the flask after step (2), continue rotary evaporating of 55° C. in water bath for 15 min to make the thin film dissolved completely, and then taking 5 ml of the resulting solution into a container of 15 ml;

(4) Putting the container into an ultrasonic pulverizer of 10 kW for 4 minutes while pumping perfluoropropane into the container to obtain an ultrasound contrast agent II5.

### Example 6-25

Example 6-25 are carried out referring to the method of Example 1, the difference is that 5 mg of the acoustic-induced deformation material PNIPAm of Example 1 is replaced with other acoustic-induced deformation material in the same weight, as shown below:

In Example 6, hematoporphyrin is used to replace PNIPAm to obtain a composition for ultrasound contrast agent I6 and an ultrasound contrast agent II6;

In Example 7, mesoporphyrin is used to replace PNIPAm to obtain a composition for ultrasound contrast agent I7 and an ultrasound contrast agent II7;

In Example 8, sodium porphyrin is used to replace PNIPAm to obtain a composition for ultrasound contrast agent I8 and an ultrasound contrast agent II8;

In Example 9, protoporphyrin is used to replace PNIPAm to obtain a composition for ultrasound contrast agent I9 and an ultrasound contrast agent II9;

In Example 10, copper protoporphyrin is used to replace PNIPAm to obtain a composition for ultrasound contrast agent I10 and an ultrasound contrast agent II10;

In Example 11, tetraphenylporphyrin tetrasulfonate is used to replace PNIPAm to obtain a composition for ultrasound contrast agent I11 and an ultrasound contrast agent II11;

In Example 12, phoeophorbide a is used to replace PNIPAm to obtain a composition for ultrasound contrast agent I12 and an ultrasound contrast agent II12;

In Example 13, Photofrin II is used to replace PNIPAm to obtain a composition for ultrasound contrast agent I13 and an ultrasound contrast agent II13;

In Example 14, ATX-70 is used to replace PNIPAm to obtain a composition for ultrasound contrast agent I14 and an ultrasound contrast agent II14;

In Example 15, ATX-S10 is used to replace PNIPAm to obtain a composition for ultrasound contrast agent I15 and an ultrasound contrast agent II15;

In Example 16, photosensory proteins is used to replace PNIPAm to obtain a composition for ultrasound contrast agent I16 and an ultrasound contrast agent II16;

In Example 17, adriamycin is used to replace PNIPAm to obtain a composition for ultrasound contrast agent I17 and an ultrasound contrast agent II17;

In Example 18, chlorin e6 is used to replace PNIPAm to obtain a composition for ultrasound contrast agent I18 and an ultrasound contrast agent II18;

In Example 19, bengal red is used to replace PNIPAm to obtain a composition for ultrasound contrast agent I19 and an ultrasound contrast agent II19;

In Example 20, erythrosin B is used to replace PNIPAm to obtain a composition for ultrasound contrast agent I20 and an ultrasound contrast agent II20;

In Example 21, curcumin is used to replace PNIPAm to obtain a composition for ultrasound contrast agent I21 and an ultrasound contrast agent II21;

In Example 22, methylene blue is used to replace PNIPAm to obtain a composition for ultrasound contrast agent I22 and an ultrasound contrast agent II22;

In Example 23, tenoxicam is used to replace PNIPAm to obtain a composition for ultrasound contrast agent I23 and an ultrasound contrast agent II23;

In Example 24, piroxicam is used to replace PNIPAm to obtain a composition for ultrasound contrast agent I24 and an ultrasound contrast agent II24;

In Example 25, water-soluble chlorin derivative PAD-S31 (13,17-bis(1-carboxypropion)carbamoylethyl-3-ethenyl-8-ethoxyiminoethylidene-7-hydroxy-2,7,12,18-tetramethyl-porphyrin sodium) (manufacturer: Photochemical Co., Ltd., Okayama, Japan) is used to replace PNIPAm to obtain a composition for ultrasound contrast agent I25 and an ultrasound contrast agent II25.

### Example 26

Referring to the method of Example 2, the difference is that the dosage of the stabilizer in the composition for ultrasound contrast agent is changed, Specifically, the stabilizer includes 18 mg of PEG1400 and 18 mg of Pluronic.

Finally, a composition for ultrasound contrast agent I26 and an ultrasound contrast agent II26 are obtained.

### Example 27

Referring to the method of Example 2, the difference is that the dosage of the stabilizer in the composition for ultrasound contrast agent is changed, Specifically, the stabilizer includes 40 mg of PEG1400 and 60 mg of Pluronic.

Finally, a composition for ultrasound contrast agent I27 and an ultrasound contrast agent II27 are obtained.

### Example 28

Referring to the method of Example 2, the difference is that the dosage of the acoustic-induced deformation material in the composition for ultrasound contrast agent is changed, Specifically, the dosage of PNIPAm is changed to 14 mg.

Finally, a composition for ultrasound contrast agent I28 and an ultrasound contrast agent II28 are obtained.

### Example 29

Referring to the method of Example 2, the difference is that the dosage of the acoustic-induced deformation material in the composition for ultrasound contrast agent is changed, Specifically, the dosage of PNIPAm is changed to 2.5 mg.

Finally, a composition for ultrasound contrast agent I29 and an ultrasound contrast agent II29 are obtained.

### Comparative Example 1

Referring to the method of Example 1, the difference is that no acoustic-induced deformation material PNIPAm is added to obtain a composition for ultrasound contrast agent IDI and an ultrasound contrast agent IID1.

### Comparative Example 2

An ultrasound contrast agent of SonoVue (manufacturer: Bracco Imaging B.V.) is purchased and denoted as an ultrasound contrast agent IID2.

### Comparative Example 3

Referring to the method of Example 1, the difference is that the dosage of PNIPAm is changed to 20 mg to obtain a composition for ultrasound contrast agent ID3 and an ultrasound contrast agent IID3.

### Test Example

The ultrasound contrast agent II1˜II26 and IID1˜IID3 obtained above are tested as follows:

(1) Stability Test

The stability of the ultrasound contrast agent in vivo is reflected by the half-life of the ultrasound contrast agent, and the longer the half-life, the higher the stability. Particularly, the test method includes (taking a rabbit as the object):

Selecting a length of vascular area in the ultrasonic image randomly after the ultrasound contrast agent is injected under the record of real-time ultrasound imaging, and recording the change of the average grey value in the area. Recording the point-in-time as t1 when the average grey value reaches a maximum A, and recording the point-in-time as t2 when the average grey value drops to 50% of the A, so the half-life of this kind of microbubbles is |t1−t2|. Note: Ensuring the concentration and dosage of the ultrasound contrast agent are same for each injection.

The measured half-life results of the ultrasound contrast agents from the Examples and Comparative Examples are recorded in Table 1, respectively.

(2) Mechanical Index Test

In vitro enriched blasting experiment is performed. Particularly, the test method includes:

Injecting the ultrasound contrast agent of the Examples and Comparative Examples with the same concentration (10⁶ pcs/mL) into the cellulose hose (inner diameter is 1 mm) with ndfeb magnet placed on one side respectively, and performing an in-vitro blasting experiment of the ultrasound contrast agent under the condition of physiological flow rate (100 mL/h). The ultrasonic imaging/blasting probe uses a linear array probe with 196 array elements and a bandwidth of 8 MHz. Setting the frequency of each examples as the characteristic response frequency (the frequency of IID1 and IID2 are set to the same as II1) for the acoustic-induced deformation material correspondingly, and controlling the mechanical index (control range is MI=0.3-1.9, stepping is 0.1) of the ultrasonic wave emitted by the ultrasonic probe through controlling the excitation pulse voltage of the ultrasonic probe. Observing the blast of the microbubbles under different mechanical index and recording the mechanical indexes respectively when the blasting rates of the ultrasound contrast agent according to each Examples and Comparative Example are greater than 90% (blasting rate=|before blasting−after blasting|/before blasting*100%), and the results are recorded in Table 1.

TABLE 1

| | | | Blasting |
|---|---|---|---|
| | | Half-life | mechanical |
| | Acoustic-induced deformation material | (min) | index |
| II1 | PNIPAm | 15.7 ± 0.5 | 0.8 |
| II2 | PNIPAm | 13.4 ± 1.4 | 0.6 |
| II3 | poly vinyl caprolactam | 13.3 ± 1.1 | 0.7 |
| II4 | poly vinyl caprolactam | 12.5 ± 1.6 | 0.6 |
| II5 | artemether (LEA) | 14.9 ± 0.9 | 0.9 |
| II6 | hematoporphyrin | 12.1 ± 0.6 | 1.2 |
| II7 | mesoporphyrin | 11.8 ± 0.9 | 1.3 |
| II8 | sodium porphyrin | 11.9 ± 1.1 | 1.2 |
| II9 | protoporphyrin | 12.3 ± 0.7 | 1.2 |
| II10 | copper protoporphyrin | 11.3 ± 1.2 | 1.3 |
| II11 | tetraphenylporphyrin tetrasulfonate | 12.6 ± 1.7 | 1.1 |
| II12 | phoeophorbide a | 10.8 ± 0.7 | 1.2 |
| II13 | Photofrin II | 11.6 ± 1.3 | 1.3 |
| II14 | ATX-70 | 11.9 ± 1.5 | 1.2 |
| II15 | ATX-S10 | 11.7 ± 1.1 | 1.2 |
| II16 | photosensory proteins | 12.8 ± 2.4 | 1.1 |
| II17 | adriamycin | 14.9 ± 1.6 | 1.2 |
| II18 | chlorin e6 | 13.4 ± 3.1 | 1.0 |
| II19 | bengal red | 12.7 ± 2.1 | 1.3 |
| II20 | erythrosin B | 12.9 ± 1.7 | 1.2 |
| II21 | curcumin | 13.6 ± 1.4 | 1.2 |
| II22 | methylene blue | 13.1 ± 2.4 | 1.3 |
| II23 | tenoxicam | 11.5 ± 1.1 | 0.9 |
| II24 | piroxicam | 13.7 ± 1.1 | 1.0 |
| II25 | PAD-S31 | 11.6 ± 1.8 | 1.1 |
| II26 | PNIPAm | 6.9 ± 2.3 | 0.4 |
| II27 | PNIPAm | 7.4 ± 1.5 | 0.5 |
| II28 | PNIPAm | 9.3 ± 2.1 | 0.5 |
| II29 | PNIPAm | 16.1 ± 0.7 | 0.9 |
| IID1 | null | 14.3 ± 0.1 | >1.9 |
| IID2 | null | 6.2 ± 2.6 | 0.8 |
| IID3 | PNIPAm | 3.2 ± 0.3 | 0.4 |

It can be seen from the test results above, compared with the comparative example, the composition for ultrasound contrast agent and the ultrasound contrast agent according to the present disclosure have both higher stability and lower mechanical index, so it is possible for the ultrasound contrast agent according to the present disclosure to act as a contrast agent and a drug delivery agent simultaneously. The preferred embodiments of the present disclosure have been described above in detail; however, the present disclosure is not limited thereto. Within the scope of the technical concept of the present disclosure, a variety of simple modifications may be made to the technical solutions of the present disclosure, including combining various technical features in any other suitable manner. These simple modifications and combinations should also be regarded as the content disclosed in the present disclosure, and they all belong to the protection scope of the present disclosure.

What is claimed is:

1. A composition for ultrasound contrast agent, wherein:
the composition for ultrasound contrast agent comprises a lipid, a stabilizer and an acoustic-induced deformation material;
relative to 100 parts by weight of the lipid, a content of the stabilizer is 20 to 100 parts by weight, and a content of the acoustic-induced deformation material is 1 to 15 parts by weight; and
the acoustic-induced deformation material is deformed under an acoustic wave of a characteristic response frequency for the acoustic-induced deformation material, and the characteristic response frequency is 0.01 MHz to 50 MHz.

2. The composition for ultrasound contrast agent according to claim 1, wherein relative to 100 parts by weight of the lipid, the content of the stabilizer is 30 to 60 parts by weight, and the content of the acoustic-induced deformation material is 3 to 12 parts by weight.

3. The composition for ultrasound contrast agent according to claim 1, wherein relative to 100 parts by weight of the lipid, the content of the stabilizer is 42 to 50 parts by weight, and the content of the acoustic-induced deformation material is 4 to 10 parts by weight.

4. The composition for ultrasound contrast agent according to claim 1, wherein the composition for ultrasound contrast agent further comprises a drug, and relative to 100 parts by weight of the lipid, the content of the drug is 2 to 20 parts by weight.

5. The composition for ultrasound contrast agent according to claim 1, wherein the characteristic response frequency of the acoustic-induced deformation material is 1 MHz to 30 MHz.

6. The composition for ultrasound contrast agent according to claim 5, wherein the characteristic response frequency of the acoustic-induced deformation material is 2 MHz to 20 MHz.

7. The composition for ultrasound contrast agent according to claim 1, wherein the acoustic-induced deformation material is any one or more selected from the group consisting of:
poly N-isopropylacrylamide, poly vinyl caprolactam, hematoporphyrin, photofrin, mesoporphyrin, sodium porphyrin, gallium porphyrin, hydrophilic chlorin derivative, protoporphyrin, copper protoporphyrin, tetraphenylporphyrin tetrasulfonate, phoeophorbide a, photosensory proteins, adriamycin, chlorin, bengal red, erythrosin B, curcumin, methylene blue, tenoxicam, piroxicam, artemether, and water-soluble chlorin derivative.

8. The composition for ultrasound contrast agent according to claim 1, wherein the lipid is a carboxylated phospholipid.

9. The composition for ultrasound contrast agent according to claim 8, wherein the lipid is any one or more selected from the group consisting of: 1,2-distearoyl-sn-glycero-3-phosphocholine (DSPC), distearoylphosphatidylethanolamine (DSPE), dipalmitoylphosphatidylcholine, 1,2-bis(diphenylphosphine)ethane (DPPE), and distearoylphosphatidylethanolamine-polyethylene glycol.

10. The composition for ultrasound contrast agent according to claim 9, wherein the lipid consists of a first lipid and a second lipid in a weight ratio of 1:0.05 to 1:0.5, wherein the first lipid is dipalmitoylphosphatidylcholine (DPPC) and/or DSPC, and the second lipid is DSPE and/or DPPE.

11. The composition for ultrasound contrast agent according to claim 1, wherein the stabilize is any one or more selected from the group consisting of: polyoxypropylene polyoxyethylene block polyether, polyethylene glycol 4000, polyethylene glycol 2000, polyethylene glycol 1400, polyethylene glycol 40s, and polysorbate-80.

12. The composition for ultrasound contrast agent according to claim 11, wherein the stabilizer is Pluronic, or the stabilizer is a combination of Pluronic with at least one of the PEG4000, PEG2000, PEG1400 and PEG40s in a weight ratio of 1:0.5 to 1:0.8.

13. An ultrasound contrast agent, wherein the ultrasound contrast agent comprises or is prepared from the composition for ultrasound contrast agent according to claim 1.

14. A method for preparing an ultrasound contrast agent, comprising:
(1) mixing the composition for ultrasound contrast agent according to claim 1 with a solvent to obtain solution A;
(2) rotary evaporating the solution A in a first water bath until a thin film is formed; and,
(3) adding a hydration solution into the material obtained by step (2) and rotary evaporating the resulting hydration solution in a second water bath until the thin film is dissolved to obtain solution B; and optionally,
(4) pumping a gas into the solution B, conducting ultrasonic cavitation to form gas-filled microbubbles.

15. The method according to claim 14, wherein the rotary evaporation temperature of the first water bath is 45° C. to 70° C., and the rotary evaporation pressure of the first water bath is 0.05 MPa to 0.5 Mpa.

16. The method according to claim 14, wherein the rotary evaporation temperature of the second water bath is 45° C. to 70° C.

17. The method according to claim 14, wherein the conditions of the ultrasonic cavitation comprise: a power of 6 kW to 12 kW, and a time of 2 min to 15 min.

18. The method according to claim 14, wherein the conditions of the ultrasonic cavitation comprise: a power of 8 kW to 12 kW, and a time of 4 min to 10 min.

19. The method according to claim 14, wherein the hydration solution is a mixture of glycerol and water or a mixture of glycerol and acid-base buffer solution.

20. The method according to claim 19, wherein the volume content of the glycerol in the hydration solution is 10% to 30%.
